(19) United States
(12) Patent Application Publication
(10) Pub. No.: US 2024/0239900 A1
(43) Pub. Date: Jul. 18, 2024

(54) A METHOD OF IMPROVING STABILITY OF AN ANTIBODY FORMULATION

(71) Applicant: DR. REDDY’S LABORATORIES LIMITED, Telangana (IN)

(72) Inventors: Murali JAYARAMAN, Kancheepuram (Dist) (IN); Swapnil VASUDEO PAKHALE, Chalisgaon Jalgaon Dist. (IN); Shrija GHOSH, Lucknow (IN); Shri Ranga Priya R. L., Srirangam Tiruchirapalli (IN); Ananya SAHA, Agartala (IN); Suman LABALA, Srikakulam (IN); Mahesh INGALE, Solapur (IN)

(21) Appl. No.: 18/289,649

(22) PCT Filed: May 5, 2022
(86) PCT No.: PCT/IN2022/050430

§ 371 Date: Nov. 6, 2023

(30) Foreign Application Priority Data
May 7, 2021 (IN) 202141020878

(51) Int. Cl. C07K 16/28; A61K 47/12; A61K 47/18; A61K 47/22; A61K 47/26

(52) U.S. Cl. CPC C07K 16/2839; A61K 47/12; A61K 47/183; A61K 47/22; A61K 47/26; C07K 2317/24

(57) ABSTRACT

The present invention discloses a stable formulation of an α4β7 antibody, wherein the formulation comprises α4β7 antibody, amino acid, a mono carboxylic acid or dicarboxylic acid or dicarboxylic acid, and surfactant. The disclosed antibody formulations are liquid high concentration formulations that are also suitable for different mode of administration (subcutaneous/intravenous). The disclosed formulations exhibit stability under various accelerated stress conditions.

### FIELD OF THE INVENTION

The present invention is related to a stable aqueous high concentration formulations of an antibody molecule, wherein the antibody present in the formulation is solubilized with excipients exhibiting physico-chemical stability. The disclosed formulations stabilizes the antibody from about 50 mg/ml to about 200 mg/ml which are suitable for intravenous or subcutaneous route of administration.

### BACKGROUND

Over the past two decades, recombinant DNA technology has led to the commercialization of many proteins, particularly antibody therapeutics. The effectiveness of these therapeutic antibodies is majorly dependent on the stability, route of administration and their dosage forms and concentrations. This in turn, necessitates therapeutic antibodies to be formulated appropriately to retain the stability and activity of a therapeutic antibody.

Formulations for each route of administration and dosage forms may be unique and, therefore, have specific requirements. Solid dosage forms, such as lyophilized powders, are generally more stable than liquid (aqueous) formulations. However, reconstitution of the lyophilized formulation requires a significant vial overfill, care in handling and involves high production cost relative to a liquid formulation. While liquid formulations are advantageous in these and are usually preferred for injectable protein therapeutics (in terms of convenience for the end user and case of preparation for the manufacturer), this form may not always be feasible given the susceptibility of proteins to denaturation, aggregation and oxidation under stresses such as temperature, pH changes, agitation etc. All of these stress factors could result in the loss of biological activity of a therapeutic protein/antibody.

In particular, high concentration liquid protein/antibody formulations exhibit lower solubility and are also susceptible to degradation and/or aggregation. This may lead to colloidal instability and function loss of the protein. Nevertheless, high concentration formulations may be desirable for subcutaneous or intravenous route of administration, as the frequency of administration and injection volume is reduced. On the other hand, specific treatment schedule and dosing might require a low concentration formulation and prefer intravenous route of administration for more predictable delivery and complete bioavailability of the therapeutic drug.

Further, monoclonal antibodies with higher surface hydrophobicity typically exhibits lower solubility. This lower solubility is due to higher intermolecular interactions and it is more prominent at high concentrations of the antibody and may lead to formation of colloidal instability or opalescence. Opalescent/turbid appearance of the solution compromises aesthetic appeal of the protein formulation and it also indicates the presence of aggregates in solution or that the system shows a tendency to undergo liquid-liquid phase separation, which indicates product's instability which may be a potential safety concern.

The excipients used for stabilizing (high concentration) formulations need to be selected considering all the above factors in entirety. Additionally, when the formulation is aimed at therapeutic use, a further concern of reduction of pain at injection site caused by any of the excipients need to be addressed. For example use of citrate in therapeutic formulations is known to cause pain at injection site or injection site pain and the pain experienced increases with increasing concentrations of such excipient/s (Shi, G. H., Pisupati, K., Parker, J. G. et al. Subcutaneous Injection Site Pain of Formulation Matrices. Pharm Res 38, 779-793 (2021). https://doi.org/10.1007/s11095-021-03047-3).

Hence, designing a formulation that is stable, and at the same time enabling soothing user experience, at high concentrations of the therapeutic protein/antibody is a significant developmental challenge. An improved formulation of existing therapeutic drug proteins is thus an ongoing requirement to improve the bio-availability and functional efficacy of the drug molecule. The present invention aims at addressing the above challenges and requirement.

### SUMMARY

The present invention discloses a high concentration formulation of an α4β7 antibody comprising, about 100 mg/ml to about 200 mg/ml antibody. In particular, the invention discloses a citrate free high concentration α4β7 antibody formulation in histidine buffer composition. The buffer composition of the present formulation comprises carboxylic acid other than citrate, and the carboxylic acid component is either a mono or dicarboxylic acid, or it's derivatives thereof.

The antibody in the said formulation is stable and maintains at least 97% of monomeric content of the antibody in the formulation even after storage for four weeks at 40° C.; at 25° C. for six months; and at 2-8° C. for six months or more. Further, the formulation composition does not require an antioxidant for maintaining it's stability and in particular, the disclosed α4β7 antibody formulations are free of citrate.

In particular, the invention discloses a method of reducing charge variants, deamidation, and/or aggregation of α4β7 antibody in its composition, by addition of acetate or lactate or succinate or glutamate, or it's derivatives thereof, to the antibody composition, during pre-formulation and/or at the formulation stage of antibody production.

The invention also discloses a method of controlling opalescence of an α4β7 antibody composition comprising addition of acetate or lactate or succinate or glutamate, or it's derivatives thereof, to the antibody composition during pre-formulation and/or at formulation steps of the antibody production to maintain the antibody in soluble form in the composition thereby maintaining opalescence. Further, the opalescence of the formulations obtained from the said process matches with reference opalescence standard (ROS) III or IV.

The invention also discloses a method to impart colloidal stability to an α4β7 antibody by formulating the antibody in a buffer composition comprising histidine buffer, and acetate or lactate or succinate or glutamate, and/or it's derivatives thereof.

The α4β7 antibody formulations of the disclosed invention exhibits stability when stored under at least one of the following conditions such as at 2-8° C. for six months; at 25° C. for six months; at 40° C. for one month. Aggregate content of the α4β7 antibody formulations is less than 2.5% when stored under the aforementioned conditions.

In addition, the invention discloses a method of controlling sub-visible particle formation in an α4β7 antibody composition, in particular in vedolizumab antibody composition, wherein the method comprises preparing the antibody composition in histidine buffer composition having a pH of 6.3 to 6.8 and comprising a mono or dicarboxylic acid, arginine, surfactant and optionally comprising sugar or amino acid. Specifically, the disclosed method controls sub-visible particles, ranging in the size of ≥10 μm, and ≥25 μm, well below the acceptable regulatory limits. Further, vedolizumab antibody formulated in the composition of histidine buffer, a monocarboxylic acid/dicarboxylic acid, arginine, and surfactant comprises lesser aggregate content of less than 2% when stored at room temperature for six months.

### DETAILED DESCRIPTION OF THE INVENTION

### Definitions

The term “about” refers to a range of values that are similar to the stated reference value and includes a range of values that fall within 20% or less, of the stated reference value.

The term “antibody” refers to a glycoprotein comprising at least two heavy (H) chains and two light (L) chains inter-connected by disulfide bonds, or an antigen-binding portion thereof. The “antibody” as used herein encompasses whole antibodies or any antigen binding fragment (i.e., “antigen-binding portion”) or fusion protein thereof.

The terms “anti-α4β7 antibody” refers to an antibody which binds to α4β7 receptor. Vedolizumab is an anti-α4β7 antibody, and the sequence of this antibody is disclosed in U.S. Pat. No. 7,147,851.

The term “stable” formulation refers to the formulation wherein the antibody therein retains its physical stability and/or chemical stability and/or biological activity upon storage.

Stability studies provides evidence of the quality of an antibody under the influence of various environmental factors during the course of time. ICH's “Q1A: Stability Testing of New Drug Substances and Products,” states that data from accelerated stability studies can be used to evaluate the effect of short-term excursions higher or lower than label storage conditions that may occur during the shipping of the antibodies.

Various analytical methods are available for measuring the physical and chemical degradation of the antibody in the pharmaceutical formulations. An antibody “retains its physical stability” in a pharmaceutical formulation if it shows substantially no signs of aggregation, precipitation and/or denaturation upon visual examination of color and/or clarity, or as measured by UV light scattering or by size exclusion chromatography. An antibody is said to “retain its chemical stability” in a pharmaceutical formulation when its shows no or minimal formation of product variants which may include variants as a result of chemical modification of antibody of interest such as deamination, oxidation etc. Analytical methods such as ion exchange chromatography and hydrophobic ion chromatography may be used to investigate the chemical product variants.

The term ‘monomer’ as used herein describes antibodies consisting of two light chains and two heavy chains. The monomer content of an antibody composition is typically analyzed by size exclusion chromatography (SEC). As per the separation principle of SEC the large molecules or molecules with high molecular weight (HMW) elute first followed by smaller or lower weight molecules. In a typical SEC profile for an antibody composition, aggregates that may include dimers, multimers, etc., elute first, followed by monomer, and the clipped antibody variants or degradants may be eluted last. In some circumstances the aggregate peak or the degradant peaks may not elute as a baseline separated peaks but instead as a shoulder or abnormal broad peaks. In order to maintain the appropriate activity of an antibody, in particular of a therapeutic antibody, it is desirable to reduce the formation of aggregate or fragmentation of products and hence control the monomer content to a target value. Ability to inhibit the formation of aggregate and degradant content as measured at various time points during stability studies may indicate the suitability of the candidate formulation for antibody of interest. TSK-GEL G3000SWXL (7.8 mm×30 cm) column from TOSCH can be used on water HPLC to perform SEC.

The term ‘main peak’ as used herein refers to the peak that elutes in abundance (major peak) during a cation exchange chromatography. The peak that elutes earlier than the main peak, during a cation exchange chromatography, with a charge that is acidic relative to the main peak is termed acidic variant peak. The peak that elutes later than the main peak, during a cation exchange chromatography, with a charge that is relatively basic than the main peak is termed as basic variant peak. The main peak content can be determined by Ion exchange chromatography (IEC). There are two modes of IEC available viz., cation and anion exchange chromatography. Negatively charged molecules bind to anion exchange resins and positively charged molecules bind to cation exchange resins. In a typical cation exchange chromatographic profile of an antibody composition acidic variants elute first followed by the main peak and thereafter lastly the basic variants will be eluted. The acidic variants are a result of antibody modifications such as deamidation of asparagine residues. The basic variants are a result of incomplete removal of C-terminal lysine residue(s). In general, in an antibody a lysine residue is present at the C-terminal end of both heavy and light chain. An antibody molecule containing lysine at both heavy and light chain is referred to as K2 variant, the antibody molecule containing lysine residue at either one of heavy and light chain is referred to as K1 variant and antibody molecule having none is K0 molecule. Carboxypeptidase B (CP-B enzyme) enzyme acts on the C-terminal lysine residues present on K2 and K1 variants and thus converting them as K0 molecules. As per circumstances of the case, the IEC analysis can be carried out for samples digested with carboxypeptidase B (CP-B) enzyme. In a typical stability study it is expected that a stable formulation leads to reduction in formation of charge variants (acidic and basic variants), during the study, and hence minimize any reduction in main peak content.

Pharmaceutically acceptable excipients refer to the additives or carriers, which may contribute to stability of the antibody in formulation. The excipients may encompass stabilizers and tonicity modifiers. Examples of stabilizers and tonicity modifiers include, but not limited to, salts, surfactants, and derivatives and combination thereof.

The term sugar/s as used herein includes sugars and sugar alcohols/polyols. Sugars can be referred to monosaccharides, disaccharides, and polysaccharides. Examples of sugars include, but are not limited to, sucrose, trehalose, glucose, dextrose, raffinose and others. Examples of sugar alcohols or polyols include, but are not limited to, mannitol, sorbitol, and others.

Surfactant refers to pharmaceutically acceptable excipients used to protect the protein formulations against various stress conditions, like agitation, shearing, exposure to high temperature etc. The suitable surfactants include but are not limited to polyoxyethylensorbitan fatty acid esters such as Tween 20™ or Tween 80™, polyoxyethylene-polyoxypropylene copolymer (e.g. Poloxamer, Pluronic), sodium dodecyl sulphate (SDS) and the like or combination thereof.

Examples of salts include, but not limited to, sodium chloride, potassium chloride, magnesium chloride, sodium thiocyanate, ammonium thiocyanate, ammonium sulfate, ammonium chloride, calcium chloride, zinc chloride and/or sodium acetate.

The term “opalescence” or “opalescent appearance” refers to the degree of turbidity detected in a solution, e.g., a protein preparation, as a function of the concentration of one or more of the components in the solution, e.g., protein and/or salt concentration. The degree of turbidity can be calculated by reference to a standard curve generated using suspensions of known turbidity. Reference standards for determining the degree of turbidity for pharmaceutical compositions can be based on the United States Pharmacopcia or European Pharmacopeia criteria. Here, in this invention to measure opalescence, first Formazine solution has been prepared by mixing equal volumes of a hydrazine sulfate solution and hexamethylenetetramine solution and then diluted to prepare various reference opalescence standards. The opalescence standards includes ROS-I, ROS-II, ROS-III and ROS-IV.

Nephelometry is a turbidometric method used to detect the presence of soluble aggregates or to indicate opalescence. The output is listed in terms of nephelometric turbidity units (NTUs).

“Pre-formulation steps” refers to any or multiple steps performed before formulating the protein into a therapeutic product. Examples of such steps include, chromatography, filtration, (ultrafiltration, sterile filtration, nano filtration, diafiltration, tangential flow filtration, depth filtration), or any other steps performed to concentrate the protein or to exchange the buffer to a different/suitable buffer. The filtration steps mentioned herein may be performed in a tangential flow filtration mode.

“Formulation steps” refers to steps which are followed after filtration/downstream chromatographic steps to prepare a drug product from the drug substance obtained from the pre-formulation steps.

The term “monocarboxylic acid” refers to an organic molecule with one carboxyl (COOH) functional group. Examples include, but not limited to, acetate, lactate.

The term “dicarboxylic acid” refers to an organic molecule with two carboxyl (COOH) functional groups. Examples include, but not limited to, succinate, glutamate.

The term “tricarboxylic acid” refers to an organic molecule with three carboxyl (COOH) functional groups. Example is citrate.

In the present invention, the sub-visible particles are measured by Micro Flow Imaging technique. Micro Flow Imaging (MFI) is an integration of microscopy, fluidics, and imaging techniques to quantify sub-visible particles and characterization of the same. Bright field images (dark image against bright background as result of reflection of the particle in the sample) are captured in successive frames as sample streams through flow cell of depth 100 μm centered in the field of view of camera of fixed magnification 5× being continuously illuminated by LED of wavelength 470 nm. The detection can be limited by particle contrast and pixels available. The measurement outcome for MFI is particle concentration (counts/mL) and shape/morphology.

Certain specific aspects and embodiments of the invention are more fully described by reference to the following examples. However, these examples should not be construed as limiting the scope of the invention in any manner.

### Detailed Description of Embodiments

The present invention discloses a pharmaceutical formulation of an α4β7 antibody comprising α4β7 antibody, in histidine buffer composition comprising a monocarboxylic acid or dicarboxylic acid, or it's derivatives thereof.

In the above mentioned embodiment, the monocarboxylic acid is acetate or lactate and dicarboxylic acid is succinate or glutamate.

In an embodiment the invention discloses a pharmaceutical formulation of an α4β7 antibody comprising, an α4β7 antibody, histidine buffer, mono or dicarboxylic acid or it's derivatives thereof.

The formulation disclosed in the invention does not require the presence of antioxidant(s). In particular, the disclosed formulations of the invention are free of citrate.

In an embodiment, the invention discloses a pharmaceutical formulation of an α4β7 antibody, comprising α4β7 antibody, histidine buffer, and any one of the excipients selected from acetate or lactate or succinate or glutamate, or it's derivatives thereof, wherein the formulation is devoid of citrate.

In an embodiment, the invention discloses citrate free high concentration vedolizumab formulation, comprising vedolizumab antibody, histidine buffer, and any one of the excipients selected from acetate or lactate or succinate or glutamate, or it's derivatives thereof.

In the above mentioned embodiments of the invention, the concentration of the α4β7/vedolizumab antibody is about 160 mg/ml to 180 mg/ml.

In an embodiment, the invention discloses a high concentration, α4β7 antibody formulation comprising about 160 mg/ml-180 mg/ml α4β7 antibody, buffer, arginine, acetate or lactate or succinate or glutamate, or it's derivatives thereof, and surfactant.

In another embodiment, the invention discloses high concentration, α4β7 antibody formulation comprising about 160 mg/ml-180 mg/ml α4β7 antibody, 50 mM histidine buffer, 5 to 50 mM acetate or lactate or succinate or glutamate, 100-125 mM arginine and 0.6 mg/ml polysorbate-80.

In the above mentioned embodiments, the α4β7 antibody formulation exhibits stability at 40° C. for two weeks.

In another embodiment, the invention discloses a method of controlling opalescence in a high concentration α4β7 antibody composition wherein the method comprises addition of a monocarboxylic acid or it's derivative to the antibody composition during pre-formulation and/or at the formulation steps of the antibody production, wherein the said method maintains the antibody in soluble form and thereby controlling the opalescence.

In the above mentioned embodiment, the monocarboxylic acid is acetate or lactate.

In another embodiment, the invention discloses a method of controlling opalescence in a high concentration α4β7 antibody composition wherein the method comprises addition of acetate or it's derivative to the antibody composition during pre-formulation and/or at the formulation steps of the antibody production, wherein the said method maintains the antibody in soluble form and thereby controlling the opalescence.

In yet another embodiment, the invention discloses a method of controlling opalescence in a high concentration α4β7 antibody composition wherein the method comprises addition of lactate or it's derivative to the antibody composition during pre-formulation and/or at the formulation steps of the antibody production, wherein the said method maintains the antibody in soluble form and thereby controlling the opalescence.

In the embodiments mentioned above, the formulation further comprises sugar, amino acid and/or a salt.

In the embodiments mentioned above, the concentration of the antibody is about 160 mg/ml or more.

In another embodiment, the invention discloses a method of controlling opalescence in a high concentration α4β7 antibody composition wherein the method comprises addition of dicarboxylic acid or it's derivate to the antibody composition during pre-formulation and/or at the formulation steps of the antibody production, wherein the said method maintains the antibody in soluble form and thereby controlling the opalescence.

In the above mentioned embodiment, the dicarboxylic acid is succinate or glutamate.

In another embodiment, the invention discloses a method of controlling opalescence in an α4β7 antibody composition wherein the method comprises addition of succinate and/or it's derivatives to the antibody composition during pre-formulation and/or at the formulation steps of the antibody production, wherein the said method maintains the antibody in soluble form and thereby controlling the opalescence.

In another embodiment, the invention discloses a method of controlling opalescence in a high concentration α4β7 antibody composition wherein the method comprises addition of glutamate or it's derivatives to the antibody composition during pre-formulation and/or at the formulation steps of the antibody production, wherein the said method maintains the antibody in soluble form and thereby controlling the opalescence.

In the above mentioned embodiments, the concentration of the antibody is about 160 mg/ml or more.

In an embodiment, the invention discloses a method of preparation of an α4β7 antibody composition, to control/reduce the sub-visible particles count in the said composition, the method comprises;
a) Obtaining a purified composition of α4β7 antibody
b) Addition of histidine buffer comprising mono or dicarboxylic acid to the antibody composition
c) Concentrating the antibody composition obtained from step b) to about 160 mg/ml, followed by,
d) Addition of arginine to the concentrated antibody composition to obtain the final antibody composition, wherein the final antibody composition has sub-visible particles count controlled/reduced, when compared with the composition that has arginine added prior to the concentration of the antibody composition, i.e., at step b).

In the above embodiment, the histidine buffer used in step b) is free of citrate.

In an embodiment, the invention discloses a method of preparation of an α4β7 antibody composition, to control/reduce the sub-visible particles count in the said composition, the method comprises;
a) Obtaining a purified composition of α4β7 antibody
b) Addition of histidine buffer comprising acetate or succinate or lactate to the antibody composition
c) Concentrating the antibody composition obtained from step b) to about 160 mg/ml, followed by,
d) Addition of arginine to the concentrated antibody composition to obtain the final antibody composition,
wherein the final antibody composition has sub-visible particles count controlled/reduced, when compared with the composition that has arginine added prior to the concentration of the antibody composition, i.e., at step b).

In the above embodiment, the histidine buffer used in step b) is free of citrate.

In an embodiment, the invention discloses a method of controlling sub-visible particle formation in an α4β7 antibody composition, wherein the method comprises preparing the antibody composition in histidine buffer, comprising a monocarboxylic acid or dicarboxylic acid or it's derivatives, amino acid and surfactant. The monocarboxylic acid is an acetate or lactate and the dicarboxylic acid is succinate.

In the above mentioned embodiment, the formulation optionally comprises sugar and/or salt.

In an embodiment, the invention discloses a method of controlling sub-visible particles formation of ≥10 μm in size in an α4β7 antibody composition, wherein the method comprises preparation of the antibody composition in a histidine buffer composition comprising acetate or lactate or succinate, arginine and surfactant.

In the above mentioned embodiments, the sub-visible particles are controlled to less than 1000 particles per ml of the antibody composition when stored at 5° C. for six months.

In an embodiment, the invention discloses a method of controlling sub-visible particles formation of ≥10 μm in size in an α4β7 antibody composition, wherein the method comprises preparation of the antibody composition in a histidine buffer composition comprising acetate or lactate, trehalose, arginine and surfactant.

In yet another embodiment, the invention discloses a method of controlling sub-visible particles formation of ≥10 μm in size in an α4β7 antibody composition, wherein the method comprises preparation of the antibody composition in a histidine buffer composition comprising acetate or lactate, glycine, arginine and surfactant, wherein the particles are controlled to less than 500 particles per ml of the antibody composition.

In an embodiment, the invention discloses a method of controlling sub-visible particles formation of ≥25 μm in size in an α4β7 antibody composition, wherein the method comprises preparation of the antibody composition in a histidine buffer composition comprising acetate or lactate or succinate, arginine and surfactant.

In the above mentioned embodiment, the sub-visible particles are controlled to less than 80 particles per ml of the antibody composition when stored at 5° C. for six months.

In the above mentioned embodiments, the formulation may optionally comprise a sugar or glycine or methionine.

In an embodiment, the invention discloses a method of reducing sub-visible particles formation of ≥25 μm in size in an α4β7 antibody composition, the method comprises preparation of the antibody composition in a histidine buffer composition comprising monocarboxylic acid or it's derivatives, arginine and surfactant, wherein the formation of particles are reduced as compared to the antibody composition formulated in histidine buffer composition comprising a tricarboxylic acid, arginine and surfactant.

In an embodiment, the invention discloses a method of controlling formation of charge variants of an α4β7 antibody in it's composition, wherein the method comprises addition of a mono or dicarboxylic acid, or it's derivatives, to the antibody composition during pre-formulation stage and/or at the formulation stage of the antibody production.

In an embodiment, the invention discloses a method of controlling formation of basic variants of an α4β7 antibody in it's composition, wherein the method comprises addition of a mono or dicarboxylic acid, or it's derivatives to the antibody composition during pre-formulation stage and/or at the formulation stage of the antibody production.

In an embodiment, the invention discloses a method of controlling formation of basic variants of an α4β7 antibody in it's composition during storage, wherein the method comprises preparation of the antibody composition in histidine buffer comprising of a mono or dicarboxylic acid, arginine, and surfactant.

In an embodiment, the invention discloses a method of controlling formation of basic variants of an α4β7 antibody in it's composition, wherein the method comprises addition of acetate to the antibody composition during pre-formulation stage and/or at the formulation stage of the antibody production.

In any of the above said embodiments of the invention, the α4β7 antibody formulation is stable with a change in the basic variants content is less than 1%, even after storage at 40° C. for two weeks.

In an embodiment, the invention discloses a method of controlling formation of acidic variants of an α4β7 antibody in it's composition during storage, wherein the method comprises preparation of the antibody composition in histidine buffer comprising of a mono or dicarboxylic acid, arginine, and surfactant.

In the above mentioned embodiments, the formulation further comprise a sugar, and/or glycine, and/or methionine, and/or sodium chloride.

In an embodiment, the invention discloses a method of controlling aggregate formation in an α4β7 antibody composition, wherein the method comprises addition of a mono or dicarboxylic acid, and/or it's derivatives, to the antibody composition during pre-formulation and/or at the formulation steps of the antibody production.

In the above mentioned embodiment, optionally one or more of the following excipients such as sugar, glycine, methionine or sodium chloride is added along with mono or dicarboxylic acid during pre-formulation and/or at the formulation steps of the antibody production.

In an embodiment, the invention discloses a method of controlling aggregate formation in an α4β7 antibody composition, wherein the method comprises preparation of the antibody in a composition comprising histidine buffer, a mono or dicarboxylic acid, arginine, and surfactant.

In the above mentioned embodiment, the aggregate content of the antibody composition is less than 2.5% when stored at room temperature for six months.

In another embodiment, the invention discloses a method of controlling aggregate formation in an α4β7 antibody composition, wherein the method comprises addition of acetate or lactate or succinate or glutamate, or it's derivatives, to the antibody composition during pre-formulation and/or at the formulation steps of the antibody production.

In another embodiment the invention discloses a method of controlling aggregation of α4β7 antibody in a pharmaceutical composition of α4β7 antibody, wherein the method comprises preparation of the antibody composition in histidine buffer comprising acetate, arginine and surfactant.

In the above mentioned embodiment, the antibody composition is stable when stored under following conditions at 2-8° C. for six months, at 25° C. for six months or at 40° C. for one month and the aggregate content is less than about 2%.

In another embodiment the invention discloses a method of controlling aggregation of α4β7 antibody in a pharmaceutical composition of α4β7 antibody, wherein the method comprises preparation of the antibody composition in histidine buffer comprising lactate, arginine and surfactant.

In the above mentioned embodiment, the antibody composition is stable when stored under following conditions at 2-8° C. for six months, at 25° C. for six months or at 40° C. for one month and the aggregate content is less than about 2%.

In another embodiment the invention discloses a method of controlling aggregation of α4β7 antibody in a pharmaceutical composition of α4β7 antibody, wherein the method comprises preparation of the antibody composition in histidine buffer comprising succinate, arginine and surfactant.

In the above mentioned embodiment, the antibody composition is stable when stored under following conditions at 2-8° C. for six months, at 25° C. for six months or at 40° C. for one month and the aggregate content is less than about 2.5%.

In any of the above mentioned embodiments, the antibody composition further include methionine.

In all of the above mentioned embodiments of the invention, the concentration of the antibody in the antibody composition or formulation is about 100 mg/ml to about 200 mg/ml. Preferably, the concentration of the antibody in the formulation 100 mg/ml, or 120 mg/ml, or 150 mg/ml or 160 mg/ml, or 170 mg/ml or 175 mg/ml or 180 mg/ml or 190 mg/ml or 195 mg/ml or 200 mg/ml.

In any of the above said embodiments of the invention, the α4β7 antibody formulation is stable and maintains at least 97% of monomeric content of the antibody, even after storage at 2-8° C. for six months; at 25° C. for six months or at 40° C. for one month.

In an embodiment, the invention discloses a method of controlling fragmentation of α4β7 antibody in a pharmaceutical composition of α4β7 antibody, wherein the method comprises preparation of the antibody composition in histidine buffer comprising acetate or lactate or succinate or glutamate, arginine and surfactant.

In the above mentioned embodiment, the antibody composition is stable when stored under following conditions at 2-8° C. for six months, at 25° C. for six months or at 40° C. for one month and the fragmentation content/low molecular weight content is less than about 1%.

In an embodiment, the invention discloses high concentration, α4β7 antibody formulation comprising about 160 mg/ml-180 mg/ml α4β7 antibody, buffer, arginine, acetate or lactate and surfactant, wherein the formulation is free of citrate.

In another embodiment, the invention discloses high concentration, α4β7 antibody formulation comprising about 160 mg/ml-180 mg/ml α4β7 antibody, 50 mM histidine buffer, 5 to 50 mM acetate or lactate, 100-125 mM arginine, and 0.6 mg/ml polysorbate-80, wherein the formulation is free of citrate.

In an embodiment, the invention discloses a pharmaceutical formulation of an α4β7 antibody comprising about 160 mg/ml α4β7 antibody, 40 mM histidine buffer, about 5-20 mM mM acetate or lactate, about 100-120 mM arginine, 10 mM methionine, and 2 mg/ml polysorbate-80, wherein the formulation is free of citrate.

In another embodiment, the invention discloses a pharmaceutical formulation of an α4β7 antibody comprising about 160 mg/ml α4β7 antibody, 40 mM histidine buffer, about 5-20 mM acetate or lactate, about 100-120 mM arginine, 5 mg/ml sorbitol, and 2 mg/ml polysorbate-80, wherein the formulation is free of citrate.

In an embodiment, the invention discloses a pharmaceutical formulation of an α4β7 antibody comprising about 160 mg/ml α4β7 antibody, 40 mM histidine buffer, about 10-30 mM succinate, about 100-120 mM arginine, 10 mg/ml trehalose, 25 mM sodium chloride, and 2 mg/ml polysorbate-80, wherein the formulation is free of citrate.

In an embodiment, the invention discloses a pharmaceutical formulation of an α4β7 antibody comprising about 160 mg/ml α4β7 antibody, 40 mM histidine buffer, about 10-30 mM succinate, about 100-120 mM arginine, 10 mg/ml trehalose, and 2 mg/ml polysorbate-80, wherein the formulation is free of citrate.

In an embodiment, the invention discloses a pharmaceutical formulation of an α4β7 antibody comprising about 160 mg/ml α4β7 antibody, 40 mM histidine buffer, about 10-30 mM succinate, about 100-120 mM arginine, glycine and 2 mg/ml polysorbate-80, wherein the formulation is free of citrate.

In an embodiment, the invention discloses a pharmaceutical formulation of an α4β7 antibody comprising about 160 mg/ml α4β7 antibody, 40 mM histidine buffer, about 10-30 mM succinate, about 100-120 mM arginine, 10 mg/ml trehalose, 10 mM methionine, and 2 mg/ml polysorbate-80, wherein the formulation is free of citrate.

In an embodiment, the invention discloses, a method of controlling viscosity of a high concentration of α4β7 antibody formulation wherein the method comprises preparation of the antibody in a composition comprising histidine buffer, a mono or dicarboxylic acid, arginine, and surfactant.

In aforementioned embodiment, viscosity of the formulation is less than 20 cP, specifically less than 14.5 cP.

In any of the above said embodiments, the viscosity of formulations is less as compared to the antibody formulated in a composition comprising histidine buffer, a tricarboxylic acid, arginine, surfactant.

In another embodiment, the invention discloses a process of imparting colloidal stability to α4β7 antibody in an α4β7 antibody formulation by addition of acetate or lactate or succinate or glutamate, or it's derivatives thereof, to the antibody formulation during pre-formulation and/or at formulation steps of the antibody production.

The α4β7 antibody formulations disclosed in the invention are biologically active.

In any of the above said embodiments, the buffer mentioned in the formulation includes organic buffer, inorganic buffer and/or combinations thereof.

In yet another embodiment of the invention, the inorganic buffer mentioned in the formulation includes phosphate buffer.

In any of the above mentioned embodiments of the invention, the pH of α4β7 antibody formulation is from 6.0-7.0; in particular the pH of the formulation is 6.3 to 6.8.

In any of the above mentioned embodiments, osmolality of the formulation is less than 500 mOsm/kg.

In any of the above mentioned embodiments, an inorganic component may be mixed with mono or dicarboxylic acids and added during any pre-formulation and/or formulations stage of antibody production.

In any of the above mentioned embodiments, the formulation of α4β7 antibody is a stable liquid (aqueous) formulation, which can be used for parenteral administration. Parenteral administration includes intravenous, subcutaneous, intra peritoneal, intramuscular administration or any other route of delivery generally considered to be falling under the scope of parenteral administration and as is well known to a skilled person.

In any of the above embodiments of the invention, the stable liquid/aqueous formulation is suitable and can be lyophilized as lyophilized powders. Further, the lyophilized formulation of α4β7 antibody can be reconstituted with appropriate diluent to achieve the liquid formulation suitable for administration.

In any of the above mentioned embodiments, the stable liquid α4β7 antibody are compatible with lyophilization process and the lyophilization process does not impact quality attributes of the antibody.

In any of the above mentioned embodiments, the α4β7 antibody includes vedolizumab.

Another aspect of the invention provides a vial, pre-filled syringe or autoinjector device, or any other suitable device comprising any of the subject formulations described herein. In certain embodiments, the aqueous formulation stored in the vial or pre-filled syringe or autoinjector device contains vedolizumab, buffer, amino acid, succinate/lactate/acetate/glutamate and surfactant.

In any of the above mentioned embodiments, α4β7 antibody formulations are visibly clear without any particles even when stored at 40° C. for two weeks.

### Examples

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

An α4β7 antibody, vedolizumab, suitable for storage in the present pharmaceutical composition is produced by standard methods known in the art. For example, vedolizumab is prepared by recombinant expression of immunoglobulin light and heavy chain genes in a mammalian host cell such as Chinese Hamster Ovary cells. The expressed vedolizumab is harvested and the crude harvest is subjected to standard downstream process steps that include purification, filtration and optionally dilution or concentration steps. For example, the crude harvest of vedolizumab may be purified using standard chromatography techniques such as affinity chromatography, ion-exchange chromatography and combinations thereof. The purified vedolizumab solution can additionally be subjected to one or more filtration steps, and the solution obtained is subjected to further formulation studies.

### Example 1: Assessment of Effect of Various Components on Opalescence of High Concentration Vedolizumab Formulations

Purified high concentration vedolizumab antibody, approximately 175 mg/ml, with about 25 mg/ml arginine in histidine buffer back ground, was obtained from downstream chromatographic steps. To this, a succinate or lactate or acetate component in histidine buffer back ground, and sodium chloride were added, to prepare sample formulations. In addition, vedolizumab at a concentration of 70 mg/ml in histidine-phosphate buffer back ground containing arginine, sodium chloride, trehalose was buffer exchanged with histidine buffer containing arginine, glutamate and sodium chloride using diafiltration and the buffer exchanged sample was concentrated upto 170 mg/ml. As a control, approved vedolizumab formulation (Entyvio®) components were used wherein approximately 100 mg/ml of purified vedolizumab in histidine buffer back ground containing 26.3 mg/ml arginine, was buffer exchanged with a composition containing histidine buffer, arginine, and citrate. Polysorbate-80 was added to all the samples above, including control. The final composition of all vedolizumab formulations are given in Table 1. The concentration of vedolizumab present in all these samples are within the range of 160 mg/ml-180 mg/ml and the concentration of histidine buffer is 50 mM. The concentration range of succinate, lactate, acetate, arginine-acetate or arginine-glutamate were in the range of 5 to 20 mM. The concentration of sodium chloride is about 25 mM and polysorbate is about 0.6 mg/ml. The pH value of all these formulations were within the range of pH 6.3 to pH 6.8.

Post formulating in excipients, all the samples were measured for their opalescence, high molecular weight species using size exclusion chromatography and charge variants using ion-exchange chromatography. To measure opalescence, various USP reference opalescence standards were prepared by diluting primary opalescence solution comprising formazin suspension having 4000 NTU ((Nephelometric Turbidity Units).

All vedolizumab formulations were then subjected for accelerated stability studies at 40° C. for two weeks. Post which, the samples were analyzed for low molecular weight (LMW) species and monomer content using size exclusion chromatography (SEC) [results are given in Table 2] and also checked for main peak content, and, basic variants using ion-exchange chromatography [Table 3], and opalescence [Table 4].

TABLE 1
Compositions of various high concentration vedolizumab
formulations prepared as per example-1

| Sample | |
|---|---|
| Name | Composition |
| Vmab- | Vedolizumab, 50 mM histidine monohydrochloride, arginine |
| Control | 26.3 mg/ml, 6.7 mg/ml sodium citrate, 0.5 mg/ml citric acid |
| | monohydrochloride, 0.6 mg/mL polysorbate 80 |
| Vmab-1 | Vedolizumab, arginine-glutamate, histidine, sodium chloride and |
| | polysorbate-80 |
| Vmab-2 | Vedolizumab, succinate, histidine, sodium chloride, arginine |
| | hydrochloride, and polysorbate-80 |
| Vmab-3 | Vedolizumab, acetate, histidine, sodium chloride, arginine |
| | hydrochloride, and polysorbate-80 |
| Vmab-4 | Vedolizumab, lactate, histidine, sodium chloride, arginine |
| | hydrochloride, and polysorbate-80 |

TABLE 2
SEC data of high concentration vedolizumab formulations prepared as per example 1

| | SEC data at 40° C. | | | | | | | | |
|---|---|---|---|---|---|---|---|---|---|
| Sample | % of LMW at 40° C. | | | % of monomer at 40° C. | | | % of HMW | | |
| Name | 0 W | 2W | Δ 2W | 0 W | 2W | Δ 2W | 0 W | 2W | Δ 2W |
| Vmab-Control | 0.1 | 0.4 | 0.3 | 99.3 | 98.6 | −0.8 | 0.6 | 1.0 | 0.4 |
| Vmab-1 | 0.03 | 0.26 | 0.2 | 99.4 | 98.1 | −1.3 | 0.56 | 1.6 | 1.1 |
| Vmab-2 | 0.06 | 0.52 | 0.5 | 99.3 | 97.9 | −1.3 | 0.65 | 1.5 | 0.9 |
| Vmab-3 | 0.14 | 0.29 | 0.2 | 99.3 | 98.6 | −0.7 | 0.56 | 1.1 | 0.6 |
| Vmab-4 | 0.06 | 0.35 | 0.3 | 99.2 | 98.3 | −0.9 | 0.71 | 1.3 | 0.6 |

W-indicates weeks,
Δ-indicates change

TABLE 3
IEX data of high concentration vedolizumab formulations
prepared as per example 1

| | IEX data at 40° C. | | | | | |
|---|---|---|---|---|---|---|
| Sample | % of basic variants at 40° C. | | | % of main peak at 40° C. | | |
| Name | 0 W | 2W | Δ 2W | 0 W | 2W | Δ 2W |
| Vmab- | 10.8 | 9.8 | −1.0 | 67.9 | 52.8 | −15.1 |
| Control | | | | | | |
| Vmab-1 | 12.6 | 23.4 | 10.8 | 66.8 | 46.2 | −20.6 |
| Vmab-2 | 12.5 | 23.7 | 11.2 | 67.2 | 51.7 | −15.5 |
| Vmab-3 | 9.2 | 8.6 | −0.6 | 66.7 | 56 | −10.7 |
| Vmab-4 | 8.87 | 14.4 | 5.5 | 65.4 | 55.8 | −9.5 |

W-indicates weeks,
Δ-indicates change

TABLE 4
Opalescence of high concentration vedolizumab
formulations prepared as per example 1

Opalescence at 40° C.

| Sample Name | 0 W | 2 W |
|---|---|---|
| Vmab-Control | ROS III | ROS III |
| Vmab-1 | ROS III | ROS IV |
| Vmab-2 | ROS III | ROS III |
| Vmab-3 | ROS IV | ROS IV |
| Vmab-4 | ROS III-IV | ROS III-IV |

All the above formulations were also checked for change in pH. It was observed that there is no change in pH of the formulations even after storage for two weeks at 40° C.

Further, all the samples were checked for visible particles.

### Examples 2: Assessment of Role of Various Excipients During Formulation and/or Pre-Formulation Steps on Stability of High Concentration Vedolizumab Formulation Under Various Thermal Stress Conditions

Purified vedolizumab antibody composition, in concentrations approximately of 3 to 4 mg/ml in phosphate buffer background, obtained from downstream chromatographic steps, was concentrated upto 30 mg/ml followed by diafiltration with 50 mM histidine buffer comprising acetate or succinate, along with arginine. Post buffer exchange, the vedolizumab antibody composition was concentrated to 160 mg/ml. To this concentrated sample, one or more of the following excipients such as trehalose or salt was added. To this, polysorbate-80 was added to prepare Vmab 6 to Vmab 13 formulation composition of Table 5. As a control, approved high concentration vedolizumab formulation (Entyvio®) components were used, wherein approximately 3-4 mg/ml of purified vedolizumab in phosphate buffer obtained from downstream chromatographic steps, and concentrated upto 30 mg/ml followed by buffer exchange with a composition containing 50 mM histidine buffer. ˜125 mM arginine and ˜25 mM citrate (Vmab-5). Post buffer exchange, the vedolizumab antibody composition was concentrated to 160 mg/ml. Polysorbate-80 was then added to the control.

Alternatively, purified vedolizumab antibody, in concentrations approximately of 3 to 4 mg/ml in phosphate buffer background, obtained from downstream chromatographic steps, was concentrated upto 30 mg/ml followed by diafiltration with 40 mM histidine buffer comprising acetate or succinate or lactate without arginine. Post buffer exchange, the vedolizumab antibody composition was concentrated to 160 mg/ml, followed by arginine addition, and one or more of the following excipients such as trehalose or sorbitol or methionine; or glycine. Polysorbate-80 was added to prepare final formulation of Vmab 15 to Vmab 26 of Table 5a. As a control, approved high concentration vedolizumab formulation (Entyvio®) components were used, wherein approximately 3-4 mg/ml of purified vedolizumab in phosphate buffer obtained from downstream chromatographic steps, and concentrated upto 30 mg/ml, followed by buffer exchange with a composition containing 50 mM histidine buffer and ˜25 mM citrate. Post buffer exchange, the vedolizumab antibody composition was concentrated to 160 mg/ml. To this, ˜125 mM arginine and polysorbate-80 was added to the control to prepare Vmab-14 of Table 5(a).

The final composition of all vedolizumab samples prepared as per example-2 given in Table 5 and Table 5(a).

Post formulating in excipients, all the samples of Table 5 and Table 5(a) were measured for their opalescence, high molecular weight species using size exclusion chromatography and charge variants using ion-exchange chromatography and viscosity using viscometer [results given in Table 15]. To measure opalescence, various USP reference opalescence standards were prepared by diluting primary opalescence solution comprising formazin suspension having 4000 NTU (Nephelometric Turbidity Units).

All the formulations of Table 5 and Table 5a were subjected for accelerated stability studies at 25° C. for six months and 40° C. for four weeks. The formulations were also subjected for real time storage conditions such as at 2-8° C. for six months. Post which, the samples were checked for sub-visible particles using Microflow Imaging technique; results are given in Table 6. And also, Results of the opalescence of the samples measured [Table 7] and osmolality of the samples measured were given in Table 7 and Table 8 respectively. r. In addition, the formulations were analyzed for high molecular weight (HMW) species; low molecular weight (LMW) species and monomer content using size exclusion chromatography (SEC) [results are given in Table 9-11] and also checked for acidic variants; main peak content, and, basic variants using ion-exchange chromatography [Table 12-14].

TABLE 5
Compositions of various high concentration vedolizumab
formulations prepared as per example-2.

| Sample | |
|---|---|
| Name | Composition |
| Vmab-5 | 160 mg/ml Vedolizumab, 50 mM histidine buffer (6.98 mg/ml |
| Control | L-histidine and 0.958 mg/ml L-histidine monohydrochloride),, |
| | arginine 26.3 mg/ml, 6.06 mg/ml sodium citrate, 1.16 mg/ml |
| | citric acid monohydro chloride, 0.6 mg/mL polysorbate 80 |
| Vmab-6 | 160 mg/ml Vedolizumab, 50 mM histidine buffer (6.98 mg/ml |
| | L-histidine and 0.958 mg/ml L-histidine monohydrochloride), |
| | 5.24 mg/ml disodium succinate, 0.07 mg/ml sodium succinate, |
| | 1.46 mg/ml sodium chloride, 10 mg/ml trehalose, and |
| | polysorbate-80 |
| Vmab-7 | 160 mg/ml Vedolizumab, 50 mM histidine buffer (6.98 mg/ml |
| | L-histidine and 0.958 mg/ml L-histidine monohydrochloride), |
| | 5.24 mg/ml disodium succinate, 0.07 mg/ml sodium succinate, |
| | 10 mg/ml trehalose, and polysorbate-80 |
| Vmab-8 | 160 mg/ml Vedolizumab, 50 mM histidine buffer (6.98 mg/ml |
| | L-histidine and 0.958 mg/ml L-histidine monohydrochloride),, |
| | 5.24 mg/ml disodium succinate, 0.07 mg/ml sodium succinate, |
| | and polysorbate-80 |
| Vmab-9 | 160 mg/ml Vedolizumab, 50 mM histidine buffer (6.98 mg/ml |
| | L-histidine and 0.958 mg/ml L-histidine monohydrochloride), |
| | 5. 24 mg/ml disodium succinate, 0.07 mg/ml sodium succinate, |
| | 1.46 mg/ml sodium chloride and polysorbate-80 |
| Vmab- | 160 mg/ml Vedolizumab, 50 mM histidine buffer (6.98 mg/ml |
| 10 | L-histidine and 0.958 mg/ml L-histidine monohydrochloride),, |
| | 30 mM acetate, 1.46 mg/ml sodium chloride and polysorbate-80 |
| Vmab- | 160 mg/ml Vedolizumab, 50 mM histidine buffer (6.98 mg/ml |
| 11 | L-histidine and 0.958 mg/ml L-histidine monohydrochloride), |
| | 30 mM acetate, 10 mg/ml trehalose, and polysorbate-80 |
| Vmab- | 160 mg/ml Vedolizumab, 50 mM histidine buffer (6.98 mg/ml |
| 12 | L-histidine and 0.958 mg/ml L-histidine monohydrochloride), |
| | 30 mM acetate, and polysorbate-80 |
| Vmab- | 160 mg/ml Vedolizumab, 50 mM histidine buffer (6.98 mg/ml |
| 13 | L-histidine and 0.958 mg/ml L-histidine monohydrochloride), |
| | 30 mM acetate, 1.46 mg/ml sodium chloride and polysorbate-80 |

TABLE 5(a)
Compositions of various high concentration vedolizumab
formulations prepared as per example-2.

| Sample | |
|---|---|
| Name | Composition |
| Vmab-14 | 160 mg/ml Vedolizumab, 50 mM histidine buffer (6.98 mg/ml |
| Control | L-histidine and 0.958 mg/ml L-histidine monohydrochloride),, |
| | arginine 26.3 mg/ml, 6.06 mg/ml sodium citrate, 1.16 mg/ml |
| | citric acid monohydro chloride, 0.6 mg/mL polysorbate 80 |
| Vmab-15 | 160 mg/ml Vedolizumab, 40 mM histidine buffer (4.34 mg/ml |
| | L-histidine and 2.51 mg/ml L-histidine hydrochloride |
| | monohydrate), 30 mM succinate, 10 mg/ml trehalose, and |
| | polysorbate-80 |
| Vmab-16 | 160 mg/ml Vedolizumab, 40 mM histidine buffer (4.34 mg/ml |
| | L-histidine and 2.51 mg/ml L-histidine hydrochloride mono- |
| | hydrate)30 mM succinate, 1.49 mg/ml methionine, and |
| | polysorbate-80 |
| Vmab-17 | 160 mg/ml Vedolizumab, 40 mM histidine buffer (4.34 mg/ml |
| | L-histidine and 2.51 mg/ml L-histidine hydrochloride mono- |
| | hydrate)30 mM succinate, 5 mg/ml sorbitol, and |
| | polysorbate-80 |
| Vmab-18 | 160 mg/ml Vedolizumab, 40 mM histidine buffer (4.34 mg/ml |
| | L-histidine and 2.51 mg/ml L-histidine hydrochloride |
| | monohydrate), 30 mM succinate, 5 mg/ml glycine, and |
| | polysorbate-80 |
| Vmab-19 | 160 mg/ml Vedolizumab, 40 mM histidine buffer (4.34 mg/ml |
| | L-histidine and 2.51 mg/ml L-histidine hydrochloride |
| | monohydrate), 15 mM acetate, 10 mg/ml trehalose, and |
| | polysorbate-80 |
| Vmab-20 | 160 mg/ml Vedolizumab, 40 mM histidine buffer (4.34 mg/ml |
| | L-histidine and 2.51 mg/ml L-histidine hydrochloride |
| | monohydrate)15 mM acetate, 1.49 mg/ml methionine, and |
| | polysorbate-80 |
| Vmab-21 | 160 mg/ml Vedolizumab, 40 mM histidine buffer (4.34 mg/ml |
| | L-histidine and 2.51 mg/ml L-histidine hydrochloride |
| | monohydrate)15 mM acetate, 5 mg/ml sorbitol, and |
| | polysorbate-80 |
| Vmab-22 | 160 mg/ml Vedolizumab, 40 mM histidine buffer (4.34 mg/ml |
| | L-histidine and 2.51 mg/ml L-histidine hydrochloride |
| | monohydrate)15 mM acetate, 5 mg/ml glycine, and |
| | polysorbate-80 |
| Vmab-23 | 160 mg/ml Vedolizumab, 40 mM histidine buffer (4.34 mg/ml |
| | L-histidine and 2.51 mg/ml L-histidine hydrochloride |
| | monohydrate)50 mM lacate, 10 mg/ml trehalose, and |
| | polysorbate-80 |
| Vmab-24 | 160 mg/ml Vedolizumab, 40 mM histidine buffer (4.34 mg/ml |
| | L-histidine and 2.51 mg/ml L-histidine hydrochloride |
| | monohydrate), 50 mM lacate, 1.49 mg/ml methionine, and |
| | polysorbate-80 |
| Vmab-25 | 160 mg/ml Vedolizumab, 40 mM histidine buffer (4.34 mg/ml |
| | L-histidine and 2.51 mg/ml L-histidine hydrochloride |
| | monohydrate), 50 mM lactate, 5 mg/ml sorbitol, and |
| | polysorbate-80 |
| Vmab-26 | 160 mg/ml Vedolizumab, 40 mM histidine buffer (4.34 mg/ml |
| | L-histidine and 2.51 mg/ml L-histidine monohydro- |
| | chloride)50 mM lactate, 5 mg/ml glycine, and |
| | polysorbate-80 |

TABLE 6
Sub visible particles data of formulations prepared as per example-2,
when stored at 5° C. for six months, and measured by MFI.

| | Sub visible particles count | |
|---|---|---|
| | ≥10 um and <100 μm | ≥25 um and <100 μm |
| Sample name | T6M | T6M |
| Vmab-5 Control | 2098 | 199 |
| Vmab-6 | 2811 | 355 |
| Vmab-7 | 1573 | 297 |
| Vmab-8 | 1945 | 242 |
| Vmab-9 | 1865 | 238 |
| Vmab-10 | 1908 | 172 |
| Vmab-11 | 3187 | 208 |
| Vmab-12 | 1194 | 156 |
| Vmab-13 | 2040 | 283 |
| Vmab-14 | 813 | 153 |
| Vmab-15 | 759 | 74 |
| Vmab-16 | 902 | 106 |
| Vmab-17 | 1108 | 94 |
| Vmab-18 | 874 | 59 |
| Vmab-19 | 617.2 | 78.1 |
| Vmab-20 | 601.6 | 58.6 |
| Vmab-21 | 648.9 | 62.5 |
| Vmab-22 | 488.6 | 78.2 |
| Vmab-23 | 811.3 | 78.0 |
| Vmab-24 | 908.2 | 78.3 |
| Vmab-25 | 547.3 | 78.2 |
| Vmab-26 | 213.2 | 15.8 |

M-indicates months;

TABLE 7
Opalescence of high concentration vedolizumab formulations prepared as per example 2

| | Visual appearance data | | | | | |
|---|---|---|---|---|---|---|
| | at 40° C. | | At 25° C. | | At 2-8° C. | |
| Sample name | T0 | T1M | T3M | T6M | T3M | T6M |
| Vmab-5 Control | ROS III | ROS III-IV | ROS III-IV | ROS III | ROS III | ROS III |
| Vmab-6 | ROS III-IV | ROS III | ROS III-IV | ROS III | ROS III-IV | ROS III-IV |
| Vmab-7 | ROS III-IV | ROS III | ROS III-IV | ROS III | ROS III | ROS III |
| Vmab-8 | ROS III | ROS III | ROS III-IV | ROS III-IV | ROS III | ROS III |
| Vmab-9 | ROS III | ROS III-IV | ROS III | ROS III-IV | ROS III | ROS III |
| Vmab-10 | ROS III-IV | ROS III-IV | ROS III | ROS III-IV | ROS III | ROS III |
| Vmab-11 | ROS III-IV | ROS III | ROS III | ROS III-IV | ROS III | ROS III |
| Vmab-12 | ROS III-IV | ROS III | ROS III-IV | ROS III-IV | ROS III | ROS III |
| Vmab-13 | ROS III-IV | ROS III | ROS III-IV | ROS III-IV | ROS III | ROS III |
| Vmab-14 | ROS III | ROS III | ROS III | ROS III | ROS III | ROS III |
| Vmab-15 | ROS III | ROS III | ROS III-IV | ROS III-IV | ROS III | ROS III |
| Vmab-16 | ROS III | ROS III | ROS III-IV | ROS III-IV | ROS III | ROS III |
| Vmab-17 | ROS III | ROS III-IV | ROS III | ROS III | ROS III | ROS III |
| Vmab-18 | ROS III | ROS III-IV | ROS III | ROS III | ROS III-IV | ROS III |
| Vmab-19 | ROS III-IV | ROS III | ROS III | ROS III | ROS III | ROS III |
| Vmab-20 | ROS III-IV | ROS III | ROS III | ROS III | ROS III | ROS III |
| Vmab-21 | ROS III-IV | ROS III-IV | ROS III | ROS III | ROS III | ROS III |
| Vmab-22 | ROS III-IV | ROS III | ROS III | ROS III | ROS III-IV | ROS III |
| Vmab-23 | ROS III-IV | ROS III-IV | ROS IV | ROS IV | ROS III-IV | >ROS IV |
| Vmab-24 | ROS III | ROS III-IV | ROS III-IV | ROS III-IV | ROS IV | >ROS IV |
| Vmab-25 | ROS III | ROS III-IV | ROS IV | ROS IV | ROS III-IV | >ROS IV |
| Vmab-26 | ROS III-IV | ROS III-IV | ROS IV | ROS IV | ROS IV | >ROS IV |

M-indicates months;
T0-represents data at zero time point;
M-indicates months;

TABLE 8
Osmolality of high concentration vedolizumab
formulations prepared as per example 2

| | Osmolality | | | | | |
|---|---|---|---|---|---|---|
| | at 40° C. | | At 25° C. | | At 2-8° C. | |
| Sample name | T0 | T1M | T3M | T6M | T3M | T6M |
| Vmab-5 Control | 369 | 402 | 380 | 469.0 | 375 | 384.0 |
| Vmab-6 | 473 | 504 | 488 | 412.5 | 484 | 484.0 |
| Vmab-7 | 415.5 | 442.0 | 431.0 | 374.5 | 436.0 | 431.0 |
| Vmab-8 | 377.0 | 413.0 | 389.0 | 436.0 | 384.0 | 378.0 |
| Vmab-9 | 436.5 | 433.5 | 448.0 | 471.5 | 447.0 | 447.0 |
| Vmab-10 | 470.3 | 501.5 | 486.0 | 421.0 | 489.0 | 480.0 |
| Vmab-11 | 420.0 | 455.5 | 432.0 | 383.5 | 428.0 | 388.5 |
| Vmab-12 | 385.0 | 409.0 | 394.5 | 435.5 | 393.0 | 440.5 |
| Vmab-13 | 436.5 | 470.0 | 447.5 | 323.0 | 441.5 | 368.5 |
| Vmab-14 | 380.0 | 388.5 | 390.0 | 383.0 | 400.0 | 386.0 |
| Vmab-15 | 421.0 | 435.0 | 424.5 | 423.0 | 434.5 | 417.0 |
| Vmab-16 | 386.0 | 396.0 | 466.5 | 498.5 | 402.5 | 418.5 |
| Vmab-17 | 421.0 | 415.0 | 427.5 | 425.5 | 415.0 | 425.5 |
| Vmab-18 | 463.0 | 466.5 | 472.5 | 452.5 | 480.5 | 480.5 |
| Vmab-19 | 368.0 | 370.5 | 371.5 | 370.5 | 378.5 | 370.5 |
| Vmab-20 | 342 | 332 | 341 | 357.0 | 341 | 348.5 |
| Vmab-21 | 368 | 360 | 362 | 369.0 | 367 | 363.5 |
| Vmab-22 | 415.0 | 415.5 | 418.0 | 420.0 | 431.0 | 423.0 |
| Vmab-23 | 339.5 | 331.5 | 347.5 | 336.0 | 342.0 | 342.0 |
| Vmab-24 | 304.5 | 307.0 | 309.5 | 309.0 | 338.5 | 308.0 |
| Vmab-25 | 330.0 | 332.0 | 344.0 | 336.0 | 351.5 | 336.0 |
| Vmab-26 | 388.5 | 395.5 | 394.0 | 394.0 | 399.5 | 390.0 |

M-indicates months;
T0-represents data at zero time point

TABLE 9
HMW content of high concentration vedolizumab
formulations of example 2, measured by SEC

| | % HMW data | | | | | | |
|---|---|---|---|---|---|---|---|
| Sample | at 40° C. | | At 25° C. | | | At 2-8° C. | |
| name | T0 | T1M | T1M | T3M | T6M | T3M | T6M |
| Vmab-5 | 0.4 | 1.2 | 0.7 | 0.9 | 1.2 | 0.5 | 0.6 |
| Control | | | | | | | |
| Vmab-6 | 0.3 | 1.3 | 0.8 | 1.2 | 1.9 | 0.5 | 0.6 |
| Vmab-7 | 0.3 | 1.4 | 0.8 | 1.3 | 2.1 | 0.6 | 0.7 |
| Vmab-8 | 0.3 | 1.5 | 0.8 | 1.2 | 2.1 | 0.6 | 0.7 |
| Vmab-9 | 0.3 | 1.4 | 0.7 | 1.2 | 2.2 | 0.6 | 0.7 |
| Vmab-10 | 0.3 | 1.5 | 0.8 | 1.3 | 1.8 | 0.5 | 0.7 |
| Vmab-11 | 0.3 | 1.4 | 0.8 | 1.2 | 1.8 | 0.5 | 0.6 |
| Vmab-12 | 0.3 | 1.4 | 0.7 | 1.2 | 1.8 | 0.5 | 0.7 |
| Vmab-13 | 0.3 | 1.4 | 0.7 | 1.3 | 2.0 | 0.5 | 0.7 |
| Vmab-14 | 0.5 | 1.0 | 0.7 | 0.9 | 1.1 | 0.6 | 0.7 |
| Vmab-15 | 0.4 | 1.5 | 0.9 | 1.6 | 2.2 | 0.7 | 0.8 |
| Vmab-16 | 0.5 | 1.3 | 0.7 | 0.9 | 0.8 | 0.6 | 0.7 |
| Vmab-17 | 0.4 | 1.6 | 0.9 | 1.6 | 2.0 | 0.7 | 0.8 |
| Vmab-18 | 0.4 | 1.5 | 0.9 | 1.3 | 1.8 | 0.7 | 0.9 |
| Vmab-19 | 0.5 | 1.4 | 0.9 | 1.4 | 1.8 | 0.7 | 0.8 |
| Vmab-20 | 0.5 | 1.2 | 0.8 | 1.1 | 1.3 | 0.7 | 0.7 |
| Vmab-21 | 0.4 | 1.4 | 0.9 | 1.3 | 2.2 | 0.7 | 0.8 |
| Vmab-22 | 0.5 | 1.3 | 0.9 | 1.3 | 1.8 | 0.7 | 0.9 |
| Vmab-23 | 0.5 | 1.4 | 0.9 | 1.3 | 1.8 | 0.7 | 0.8 |
| Vmab-24 | 0.5 | 1.2 | 0.8 | 1.0 | 1.2 | 0.6 | 0.8 |
| Vmab-25 | 0.5 | 1.4 | 0.9 | 1.3 | 1.8 | 0.7 | 0.8 |
| Vmab-26 | 0.5 | 1.4 | 0.9 | 1.2 | 1.7 | 0.7 | 0.9 |

M-indicates months;
T0-represents data at zero time point

TABLE 10
Monomer content of high concentration vedolizumab
formulations of example 2, measured by SEC

| | % Monomer Content | | | | | | |
|---|---|---|---|---|---|---|---|
| Sample | at 40° C. | | At 25° C. | | | At 2-8° C. | |
| name | T0 | T1M | T1M | T3M | T6M | T3M | T6M |
| Vmab-5 | 99.6 | 98.3 | 99.1 | 98.6 | 98.1 | 99.2 | 99.3 |
| Control | | | | | | | |
| Vmab-6 | 99.6 | 98.1 | 99.1 | 98.3 | 97.3 | 99.1 | 99.2 |
| Vmab-7 | 99.7 | 97.9 | 99.0 | 98.2 | 97.2 | 99.1 | 99.0 |
| Vmab-8 | 99.6 | 97.9 | 99.0 | 98.2 | 97.2 | 99.1 | 99.1 |
| Vmab-9 | 99.6 | 97.9 | 99.1 | 98.3 | 97.1 | 99.2 | 99.1 |
| Vmab-10 | 99.7 | 97.8 | 99.0 | 98.0 | 97.3 | 99.1 | 99.1 |
| Vmab-11 | 99.6 | 97.9 | 99.0 | 98.2 | 97.4 | 99.1 | 99.2 |
| Vmab-12 | 99.6 | 98.0 | 99.1 | 98.2 | 97.5 | 99.1 | 99.1 |
| Vmab-13 | 99.6 | 97.9 | 99.1 | 98.2 | 97.2 | 99.1 | 99.1 |
| Vmab-14 | 99.5 | 98.3 | 99.2 | 98.7 | 98.4 | 99.2 | 99.2 |
| Vmab-15 | 99.5 | 97.8 | 98.9 | 98.0 | 97.2 | 99.1 | 99.1 |
| Vmab-16 | 99.5 | 98.0 | 99.1 | 98.7 | 98.1 | 99.2 | 99.2 |
| Vmab-17 | 99.5 | 97.7 | 98.9 | 98.0 | 97.4 | 99.1 | 99.1 |
| Vmab-18 | 99.5 | 97.8 | 98.9 | 98.3 | 97.6 | 99.1 | 99.0 |
| Vmab-19 | 99.5 | 97.9 | 98.9 | 98.2 | 97.6 | 99.1 | 99.1 |
| Vmab-20 | 99.5 | 98.1 | 99.0 | 98.6 | 98.1 | 99.2 | 99.2 |
| Vmab-21 | 99.5 | 97.9 | 98.9 | 98.3 | 97.2 | 99.1 | 99.1 |
| Vmab-22 | 99.5 | 98.0 | 98.9 | 98.3 | 97.5 | 99.2 | 99.0 |
| Vmab-23 | 99.4 | 98.0 | 98.9 | 98.3 | 97.5 | 99.1 | 99.0 |
| Vmab-24 | 99.4 | 98.3 | 99.1 | 98.6 | 98.1 | 99.2 | 99.1 |
| Vmab-25 | 99.4 | 98.0 | 98.9 | 98.3 | 97.5 | 99.1 | 99.0 |
| Vmab-26 | 99.4 | 98.0 | 98.9 | 98.3 | 97.6 | 99.1 | 99.0 |

M-indicates months;
T0-represents data at zero time point

TABLE 11
Low molecular weight (LMW) content of high concentration
vedolizumab formulations of example 2, measured by SEC

| | % LMW | | | | | | |
|---|---|---|---|---|---|---|---|
| Sample | at 40° C. | | At 25° C. | | | At 2-8° C. | |
| name | T0 | T1M | T1M | T3M | T6M | T3M | T6M |
| Vmab-5 | 0.0 | 0.6 | 0.2 | 0.5 | 0.7 | 0.2 | 0.2 |
| Control | | | | | | | |
| Vmab-6 | 0.1 | 0.6 | 0.2 | 0.5 | 0.7 | 0.3 | 0.2 |
| Vmab-7 | 0.0 | 0.7 | 0.2 | 0.5 | 0.8 | 0.3 | 0.3 |
| Vmab-8 | 0.1 | 0.7 | 0.2 | 0.5 | 0.8 | 0.3 | 0.2 |
| Vmab-9 | 0.1 | 0.7 | 0.2 | 0.5 | 0.8 | 0.3 | 0.2 |
| Vmab-10 | 0.0 | 0.7 | 0.2 | 0.7 | 0.8 | 0.4 | 0.2 |
| Vmab-11 | 0.1 | 0.7 | 0.2 | 0.6 | 0.8 | 0.4 | 0.2 |
| Vmab-12 | 0.1 | 0.7 | 0.2 | 0.6 | 0.8 | 0.3 | 0.3 |
| Vmab-13 | 0.1 | 0.7 | 0.2 | 0.6 | 0.8 | 0.4 | 0.3 |
| Vmab-14 | 0.1 | 0.6 | 0.1 | 0.4 | 0.6 | 0.2 | 0.1 |
| Vmab-15 | 0.0 | 0.7 | 0.2 | 0.4 | 0.6 | 0.2 | 0.1 |
| Vmab-16 | 0.0 | 0.7 | 0.2 | 0.5 | 1.0 | 0.2 | 0.1 |
| Vmab-17 | 0.0 | 0.7 | 0.2 | 0.4 | 0.7 | 0.2 | 0.1 |
| Vmab-18 | 0.1 | 0.8 | 0.2 | 0.4 | 0.6 | 0.2 | 0.1 |
| Vmab-19 | 0.1 | 0.7 | 0.2 | 0.4 | 0.6 | 0.2 | 0.1 |
| Vmab-20 | 0.1 | 0.7 | 0.2 | 0.4 | 0.6 | 0.2 | 0.1 |
| Vmab-21 | 0.0 | 0.7 | 0.2 | 0.4 | 0.6 | 0.2 | 0.1 |
| Vmab-22 | 0.1 | 0.7 | 0.2 | 0.4 | 0.6 | 0.2 | 0.1 |
| Vmab-23 | 0.1 | 0.6 | 0.2 | 0.5 | 0.7 | 0.2 | 0.2 |
| Vmab-24 | 0.1 | 0.6 | 0.2 | 0.5 | 0.7 | 0.2 | 0.2 |
| Vmab-25 | 0.1 | 0.6 | 0.2 | 0.5 | 0.7 | 0.2 | 0.2 |
| Vmab-26 | 0.1 | 0.6 | 0.2 | 0.5 | 0.7 | 0.2 | 0.2 |

M-indicates months;
T0-represents data at zero time point

TABLE 12
Acidic variants of high concentration vedolizumab
formulations of example 2, measured by IEX

| | %-Acidic variants | | | | | | |
|---|---|---|---|---|---|---|---|
| Sample | at 40° C. | | At 25° C. | | | At 2-8° C. | |
| name | T0 | T1M | T1M | T3M | T6M | T3M | T6M |
| Vmab-5 | 20.9 | 35.7 | 21.1 | 28.1 | 35.1 | 20.9 | 19.2 |
| Control | | | | | | | |
| Vmab-6 | 21.4 | 36.8 | 21.8 | 28.8 | 37.7 | 21.3 | 19.8 |
| Vmab-7 | 22.0 | 37.0 | 21.9 | 28.4 | 37.6 | 21.4 | 20.5 |
| Vmab-8 | 23.6 | 38.2 | 22.5 | 27.7 | 37.6 | 21.6 | 19.4 |
| Vmab-9 | 22.0 | 37.5 | 23.3 | 28.0 | 38.3 | 21.6 | 19.9 |
| Vmab-10 | 21.9 | 37.9 | 22.4 | 29.6 | 37.5 | 21.7 | 19.9 |
| Vmab-11 | 22.3 | 37.9 | 22.0 | 31.0 | 36.7 | 21.5 | 20.0 |
| Vmab-12 | 21.8 | 37.5 | 21.5 | 31.4 | 38.0 | 21.9 | 20.2 |
| Vmab-13 | 24.0 | 37.7 | 22.0 | 31.8 | 38.3 | 22.0 | 20.4 |
| Vmab-14 | 17.4 | 37.2 | 18.1 | 24.9 | 34.51 | 16.7 | 18.3 |
| Vmab-15 | 20.3 | 43.3 | 22.6 | 30.7 | 40.41 | 20.7 | 21.6 |
| Vmab-16 | 20.6 | 42.2 | 23.2 | 41.8 | 46.04 | 20.4 | 22.0 |
| Vmab-17 | 20.6 | 42.8 | 23.1 | 29.9 | 40.12 | 20.6 | 22.1 |
| Vmab-18 | 20.9 | 43.3 | 23.3 | 30.8 | 40.36 | 20.5 | 22.3 |
| Vmab-19 | 21.4 | 40.3 | 23.1 | 29.2 | 38.18 | 20.5 | 21.7 |
| Vmab-20 | 22.2 | 40.1 | 23.0 | 28.7 | 37.55 | 20.5 | 21.9 |
| Vmab-21 | 21.5 | 40.2 | 23.2 | 29.2 | 38.82 | 20.7 | 21.8 |
| Vmab-22 | 22.0 | 41.0 | 22.5 | 28.9 | 39.52 | 20.8 | 22.4 |
| Vmab-23 | 18.6 | 39.3 | 21.1 | 26.8 | 36.2 | 17.6 | 17.6 |
| Vmab-24 | 19.1 | 39.5 | 21.3 | 26.6 | 35.4 | 16.9 | 17.4 |
| Vmab-25 | 18.9 | 39.6 | 21.3 | 26.8 | 35.9 | 17.5 | 17.5 |
| Vmab-26 | 18.9 | 40.7 | 21.8 | 27.5 | 36.6 | 17.3 | 17.7 |

M-indicates months;
T0-represents data at zero time point

TABLE 13
Main peak content of high concentration vedolizumab
formulations of example 2, measured by IEX

| | % Main peak Content | | | | | | |
|---|---|---|---|---|---|---|---|
| Sample | at 40° C. | | At 25° C. | | | At 2-8° C. | |
| name | T0 | T1M | T1M | T3M | T6M | T3M | T6M |
| Vmab-5 | 72.6 | 57.6 | 70.1 | 64.6 | 55.6 | 70.0 | 73.2 |
| Control | | | | | | | |
| Vmab-6 | 72.5 | 57.1 | 69.9 | 64.6 | 72.1 | 70.5 | 55.3 |
| Vmab-7 | 72.0 | 57.1 | 70.1 | 65.1 | 72.2 | 70.2 | 73.1 |
| Vmab-8 | 70.6 | 56.0 | 69.4 | 63.9 | 57.9 | 70.1 | 73.0 |
| Vmab-9 | 72.2 | 56.5 | 68.8 | 64.0 | 70.7 | 70.2 | 55.4 |
| Vmab-10 | 72.3 | 55.8 | 70.2 | 62.8 | 72.2 | 69.8 | 73.0 |
| Vmab-11 | 71.9 | 55.9 | 70.5 | 61.3 | 57.5 | 70.3 | 56.9 |
| Vmab-12 | 72.4 | 56.2 | 71.1 | 62.1 | 55.3 | 69.5 | 72.8 |
| Vmab-13 | 70.4 | 56.3 | 70.7 | 61.8 | 55.5 | 69.7 | 56.0 |
| Vmab-14 | 71.5 | 54.8 | 72.7 | 67.7 | 58.4 | 75.1 | 72.1 |
| Vmab-15 | 71.8 | 50.6 | 70.5 | 65.0 | 54.9 | 72.3 | 71.0 |
| Vmab-16 | 71.3 | 51.2 | 72.2 | 56.2 | 29.87 | 73.1 | 71.0 |
| Vmab-17 | 71.7 | 50.7 | 70.4 | 65.6 | 55.2 | 72.6 | 70.6 |
| Vmab-18 | 71.1 | 50.1 | 70.2 | 65.4 | 55.3 | 72.9 | 70.4 |
| Vmab-19 | 70.6 | 52.3 | 70.1 | 66.8 | 56.8 | 72.8 | 71.2 |
| Vmab-20 | 69.7 | 52.3 | 70.4 | 67.4 | 58.1 | 72.8 | 71.0 |
| Vmab-21 | 70.3 | 52.2 | 69.9 | 67.0 | 56.4 | 72.7 | 71.1 |
| Vmab-22 | 69.5 | 51.3 | 70.4 | 66.4 | 55.8 | 72.6 | 70.3 |
| Vmab-23 | 72.0 | 51.8 | 70.2 | 66.1 | 57.7 | 73.3 | 74.0 |
| Vmab-24 | 71.8 | 51.9 | 70.2 | 66.2 | 58.3 | 74.3 | 74.5 |
| Vmab-25 | 72.3 | 51.9 | 70.2 | 66.0 | 57.8 | 73.9 | 74.4 |
| Vmab-26 | 72.4 | 50.9 | 69.7 | 65.5 | 57.2 | 73.8 | 74.2 |

M-indicates months;
T0-represents data at zero time point

TABLE 14
Basic variants of high concentration vedolizumab
formulations of example 2, measured by IEX

| | % Basic variants | | | | | | |
|---|---|---|---|---|---|---|---|
| Sample | at 40° C. | | At 25° C. | | | At 2-8° C. | |
| name | T0 | T1M | T1M | T3M | T6M | T3M | T6M |
| Vmab-5 | 6.5 | 6.7 | 8.8 | 7.3 | 6.4 | 9.1 | 7.1 |
| Control | | | | | | | |
| Vmab-6 | 6.1 | 6.0 | 8.3 | 6.6 | 7.0 | 8.2 | 11.3 |
| Vmab-7 | 6.0 | 5.9 | 8.1 | 6.5 | 11.2 | 8.4 | 7.7 |
| Vmab-8 | 5.9 | 5.8 | 8.0 | 8.3 | 6.0 | 8.3 | 6.9 |
| Vmab-9 | 5.9 | 6.0 | 7.9 | 8.0 | 6.0 | 8.1 | 6.6 |
| Vmab-10 | 5.8 | 6.3 | 7.4 | 7.6 | 5.4 | 8.6 | 7.0 |
| Vmab-11 | 5.7 | 6.2 | 7.5 | 7.7 | 6.8 | 8.2 | 7.5 |
| Vmab-12 | 5.8 | 6.3 | 7.4 | 6.5 | 4.8 | 8.6 | 6.9 |
| Vmab-13 | 5.6 | 6.1 | 7.4 | 6.4 | 11.2 | 8.3 | 6.9 |
| Vmab-14 | 8.2 | 8.0 | 9.2 | 7.42 | 7.0 | 8.1 | 9.7 |
| Vmab-15 | 8.1 | 6.2 | 6.9 | 4.3 | 4.7 | 7.0 | 7.5 |
| Vmab-16 | 10.8 | 6.7 | 4.6 | 2.0 | 24.1 | 6.5 | 7.0 |
| Vmab-17 | 8.1 | 6.5 | 6.5 | 4.5 | 4.7 | 6.8 | 7.3 |
| Vmab-18 | 7.7 | 6.6 | 6.5 | 3.8 | 4.3 | 6.6 | 7.3 |
| Vmab-19 | 8.1 | 7.4 | 6.9 | 4.1 | 5.0 | 6.7 | 7.1 |
| Vmab-20 | 8.0 | 7.7 | 6.7 | 3.9 | 4.3 | 6.6 | 7.2 |
| Vmab-21 | 8.2 | 7.6 | 6.9 | 3.9 | 4.8 | 6.6 | 7.2 |
| Vmab-22 | 8.5 | 7.7 | 7.1 | 4.7 | 4.6 | 6.6 | 7.3 |
| Vmab-23 | 9.4 | 8.9 | 8.7 | 7.1 | 6.2 | 9.1 | 8.4 |
| Vmab-24 | 9.1 | 8.7 | 8.6 | 7.3 | 6.3 | 8.9 | 8.0 |
| Vmab-25 | 8.8 | 8.5 | 8.5 | 7.3 | 6.3 | 8.6 | 8.1 |
| Vmab-26 | 8.7 | 8.5 | 8.5 | 7.1 | 6.1 | 8.9 | 8.1 |

M-indicates months;
T0-represents data at zero time point

TABLE 15
Viscosity of high concentration vedolizumab
formulations of example 2.

| Sample name | T0 |
|---|---|
| Vmab-5 Control | 14.6 |
| Vmab-6 | 13.4 |
| Vmab-7 | 13.8 |
| Vmab-8 | 13.3 |
| Vmab-9 | 14.1 |
| Vmab-10 | 14.8 |
| Vmab-11 | 15.4 |
| Vmab-12 | 15.3 |
| Vmab-13 | 14.3 |
| Vmab-14 | 15.4 |
| Vmab-15 | 13.8 |
| Vmab-16 | 11.8 |
| Vmab-17 | 12.3 |
| Vmab-18 | 12.6 |
| Vmab-19 | 12.6 |
| Vmab-20 | 12.6 |
| Vmab-21 | 12.9 |
| Vmab-22 | 12.7 |
| Vmab-23 | 12.8 |
| Vmab-24 | 12.3 |
| Vmab-25 | 13.4 |
| Vmab-26 | 13.7 |

T0-represents data at zero time point

Further, concentration of the antibody present in the formulations of example-2 is measured after thermal stress conditions and it was observed that there is no significant change in the concentration of the antibody even after storage at room temperature for six months and also at 40° C. for one month. In addition, pH of the samples were measured at different time points during thermal stress stability studies and pH of the formulations were within the range of 6.3 to 6.8

1. A pharmaceutical formulation of a high concentration α4β7 antibody comprising an α4β7 antibody, histidine buffer, and mono or dicarboxylic acid, or its derivatives thereof.

2. A pharmaceutical formulation of high concentration vedolizumab comprising 160 mg/ml vedolizumab 40 mM histidine buffer having a pH of 6.3 to 6.8, 5-50 mM acetate or lactate or succinate or glutamate, about 120 mM arginine and 120 mg/ml polysorbate-80, wherein the formulation is free of citrate.

3. A method of controlling sub-visible particles formation in an α4β7 antibody, wherein the method comprises preparation of the antibody in a composition comprising histidine buffer, mono or a dicarboxylic acid, arginine and surfactant.

4. A method of controlling formation of charge variants or aggregates or fragmentation of an antibody in a high concentration α4β7 antibody composition, wherein the method comprises preparation of the antibody in a composition comprising histidine buffer, mono or dicarboxylic acid, arginine, and surfactant.

5. A method of controlling opalescence in a high concentration α4β7 antibody composition wherein the method comprises addition of histidine buffer comprising a mono or dicarboxylic acid to the antibody composition during pre-formulation and/or at the formulation steps of the antibody production, wherein the said method maintains the antibody in soluble form and thereby controlling the opalescence.

6. The formulation of claim 1, which further comprises trehalose or sorbitol or methionine or glycine.

7. (canceled)

8. The formulation as claimed in claim 1, wherein the antibody concentration ranges from about 160 mg/ml to 180 mg/ml.

9. A method of preparation of an α4β7 antibody composition, to control or reduce the sub-visible particles count in the said composition, the method comprises;
a) obtaining a purified composition of α4β7 antibody
b) addition of histidine buffer comprising a mono or dicarboxylic acid to the antibody composition
c) concentrating the antibody composition obtained from step b), to about 160 mg/ml, followed by,
d) addition of arginine to the concentrated antibody composition to obtain a final antibody composition,
wherein the final antibody composition has sub-visible particles count controlled or reduced.

10. The antibody formulation of claim 1, wherein the monocarboxylic acid is acetate or lactate, and dicarboxylic acid is succinate or glutamate.

11. The formulation of claim 2, which further comprises trehalose or sorbitol or methionine or glycine.

12. The formulation as claimed in claim 2, wherein the antibody concentration ranges from about 160 mg/ml to 180 mg/ml.
